(19) United States
(12) Patent Application Publication
(10) Pub. No.: US 2024/0288595 A1
(43) Pub. Date: Aug. 29, 2024

(54) METHOD AND SYSTEM FOR HIGH PHOTON ENERGIES IMAGING

(71) Applicant: BAR ILAN UNIVERSITY, Ramat Gan (IL)

(72) Inventors: Eliahu COHEN, Rehovot (IL); Sharon SHWARTZ, Kiryat Ono (IL); Adi BEN-YEHUDA, Haifa (IL); Or SEFI, Petach-Tikva (IL); Yishay KLEIN, Amatzia (IL); Shimon SUKHOLUSKI, Kiryat Gat (IL)

(73) Assignee: BAR ILAN UNIVERSITY, Ramat Gan (IL)

(21) Appl. No.: 18/571,674

(22) PCT Filed: Jul. 27, 2022
(86) PCT No.: PCT/IL2022/050814

§ 371 Date: Dec. 18, 2023

Related U.S. Application Data
(60) Provisional application No. 63/226,195, filed on Jul. 28, 2021.

(51) Int. Cl. G01T 1/29; G01N 23/04; G01N 23/20066; G01N 23/203

(52) U.S. Cl. CPC G01T 1/295; G01N 23/04; G01N 23/20066; G01N 23/203

(57) ABSTRACT

A masking assembly is presented for use in inspecting a region of interest by creating structured high photon energy radiation to interact with the region of interest. The masking assembly comprises at least one mask structure, the mask structure having a predetermined effective thickness t, and comprising a mask pattern formed by an arrangement of spaced-apart features of absorption properties with respect to said high photon energy radiation different from spaces between said features. The arrangement of said features along a lateral dimension of the pattern defines a predetermined effective pattern size P. The features have a characteristic lateral size/providing a substantially high aspect ratio t/l.

### TECHNOLOGICAL FIELD

The present invention is in the field of high photon energies imaging techniques and relates to X-ray or gamma-ray imaging system and method utilizing structured radiation for inspecting samples.

### BACKGROUND ART

References considered to be relevant as background to the presently disclosed subject matter are listed below:
1. A. Geva, Y. Y. Schechner, Y. Chernyak, R. Gupta, Proc. European Conf. Computer Vision (ECCV 2018).
2. T. J. Lane and D. Ratner, Opt. Express 28, 5898 (2020).
3. M. J. Padgett and R. W. Boyd, Philos. Trans. R. Soc. A 375, 20160233 (2017).
4. A.-X. Zhang, Y.-H. He, L.-A. Wu, L.-M. Chen, and B.-B Wang, Optica 5, 374 (2018).
5. D. Pelliccia, A. Rack, M. Scheel, V. Cantelli, and D. M. Paganin, Phys. Rev. Lett. 117, 113902 (2016).
6. H. Yu et al., Phys. Rev. Lett. 117, 113901 (2016).
7. A. Schori, and S. Shwartz, Opt. Express 25, 14822 (2017).
8. A. M. Kingston et al., Optica 5, 1516 (2018).
9. A. Schori, D. Borodin, K. Tamasaku, and S. Shwartz, Phys. Rev. A 97, 063804 (2018).
10. Y. Klein, A. Schori, I. P. Dolbnya, K. Sawhney, S. Shwartz, Opt. Exp. 27, 3284 (2019).
10. N. D. Hardy and J. H. Shapiro, Phys. Rev. A 84, 063824 (2011).
11. F. Wang, H. Wang, H. Wang, G. Li, and G. Situ, Opt. Express 27, 25560 (2019).

Acknowledgement of the above references herein is not to be inferred as meaning that these are in any way relevant to the patentability of the presently disclosed subject matter.

### BACKGROUND

X-ray and gamma-ray imaging methods are used for numerous applications in a variety of areas ranging from basic science to medicine, industry, and security. The main advantage of this very high photon energy radiation for imaging is its unique capability to penetrate through surfaces, which are opaque to other commonly used wavelengths, such as visible and infrared. However, despite the existence of many instruments and devices, which are based on X-ray and gamma-ray imaging, there are several physical limitations that restrict the resolution and contrast of these techniques.

The main fundamental challenge is the absence of high-quality lenses, thus high photon energy microscopes that operate like microscopes at optical wavelengths are devices that are very challenging to implement. There is a very fundamental physical reason for this challenge: the refractive index of any material in the high-photon energy range is nearly unity, thus the refractive index contrast between the lens material and the environment is tiny, leading to a very small magnification, which is impractical for imaging applications. From similar considerations, mirrors require very small grazing angles, which again are impractical. Moreover, the requirement for lens and mirror surfaces with roughness better than the wavelength is not possible since the wavelength is shorter than the size of one atom, thus phase distortions are induced. Lenses with very limited capabilities, which are also insufficient for practical applications, have been presented at photon energies up to about 150 keV but not at higher photon energies. In the absence of lenses, the spatial information for measurements at very high-photon energies is retrieved by using pixelated detectors and by measuring the intensity variation from one pixel to another. It is clear that with these detectors the resolution is limited by the pixel size. However, absorption is also weak in this photon energy range, which implies that the efficiency of the direct detection is small. To overcome this challenge, scintillation detectors are used. In those detectors, the photons are converted by a scintillation screen to visible photons, which are then detected by a visible-radiation-sensitive detector.

Nevertheless, even with scintillators, the efficiency depends on the absorption of the scintillation screen, thus the requirement for sufficient detection efficiency leads to firm requirements on the minimal thickness of the screen. Since the blurring introduced by the scintillation screen increases with the screen thickness, the resolution of the image is limited by the screen thickness. This fundamental challenge becomes more severe at higher photon energies as the absorption coefficient decreases.

Another challenge is also inherently related to the physics of X-ray and gamma-rays, namely, these are ionizing forms of radiation, threatening both the operators (e.g. technicians, surgeons) and subjects (e.g. patients, radiation-sensitive materials). Therefore, an important aim is to reduce the absorbed radiation power. Practical challenges include the current cost and size of X-ray and gamma-ray imaging devices.

### GENERAL DESCRIPTION

There is a need in the art for a novel approach for inspecting samples by imaging using structured X-ray or gamma-ray radiation.

Traditionally, there are two approaches for active imaging: full field imaging where the whole object is measured by a two-dimensional (2D) detector and a scanning method in which a focused beam is used, and the sample is raster scanned. Another technique utilizes structured illumination, which enables to introduce spatial variations of intensity or phase, usually by a spatial light modulator (in the visible regime) or diffuser (in higher photon energies), and the image is acquired by measuring various realizations of the intensity or phase structures. This method has several potential advantages over the traditional methods. First, in structured illumination the resolution is limited by the variation of the intensity, which is introduced by the diffuser and not by the pixel size of the detector, thus higher resolution than that of standard 2D detectors can be achieved. For wavelength ranges such as X-rays and gamma-rays the insertion of a diffuser into the beam's path is vital due to the absence of high-quality, non-expensive lenses which could be used for full-field imaging or high-quality, computer controlled spatial light modulators which could be used for ghost imaging. Furthermore, since most objects/samples are either sparse or can be transformed into a basis where they are sparse, the number of required realizations can be made significantly lower than the data points with raster scanning. When combined with intensity correlation measurements, the flux of radiation on the object can be substantially reduced.

Ghost Imaging (GI) is an imaging scheme, which utilizes either classically or quantumly correlated radiation. This technique has received considerable attention throughout the years mainly with regards to visible radiation. The key idea of GI is to use two beams with identical intensity fluctuations. One of the beams is measured by a two-dimensional detector, but that beam does not interact with the object. The second beam interacts with the object and then it is collected by a single-pixel detector that has no spatial resolution.

The image is reconstructed by correlating the two measured intensities at the two detectors for many different realizations of input intensity fluctuation. Alternatively, and more practically, it is possible to perform two series of measurements; in the first step the diffuser is scanned without the object and the intensity distribution is recorded by using a high-resolution imaging system that included a pixelated detector or a pencil beam. In the second step the object is inserted, and the pixelated detector is replaced by a single-pixel detector. The diffuser is scanned again for the same realization as in the first step. Finally, the two series of data are correlated for the reconstruction of the image. This method is often called computational ghost imaging.

In recent years, several groups have demonstrated the possibility to utilize GI techniques for X-ray imaging and suggested that it can be used for dose reduction. GI was traditionally performed on transmissive objects, but it was shown that it can be applied also to reflective scenarios where the single-pixel detector is positioned next to the illumination source as in remote sensing applications. It was also shown that reflective GI through turbulent media can be properly addressed.

For at least the challenges described above, there remains a need for a radically new method that can surmount them and lead to new capabilities for high-photon energy measurements in general and for imaging in particular.

The technique of the present invention encompasses methods and systems for high photon energies imaging and their use for dose reduction in medical imaging and non-destructive imaging, for resolution improvement, and for reducing scattering effects or for a single-sided operation in X-ray and gamma-ray measurements. The present invention utilizes detection of elastic and inelastic (Compton) scattering. In this connection, it should be noted that although such Compton scattering often reduces the contrast of the reconstructed image and leads to image blurring and reduction in the sensitivity of the measurements, the technique of the present invention utilizes a specifically designed structured radiation enabling to significantly improve the image resolution extracted from detection of Compton scattering.

Typically, the X-ray or gamma-ray source may be X-ray tube, radioactive nuclei, positron-electron annihilation, or inverse Compton scattering source. In the description below, the radiation source and emitted radiation are referred to as “X-ray source” and “X-ray radiation”.

The present invention provides a novel design of a mask assembly and its operation to sequentially produce mutually different spatially encoded X-ray beams interacting with a region of interest being inspected. The mask structure (which may for example be configured as a plate-like structure) has a predetermined effective thickness t, and is formed with a pattern of an arrangement of spaced-apart features of different absorption of the high photon energy radiation used in the inspection as compared to that of spaces between them. The pattern has a predetermined pattern size defined by the arrangement of these features along a lateral dimension of the pattern. The features have a characteristic lateral size l providing a substantially high aspect ratio t/l.

Typically, the mask pattern may be in the form of a 3D printed structured and/or spongy or porous materials; or the mask may comprise structures created by laser ablation. In some embodiments, the mask pattern is optimized using advanced algorithms such as compressed sensing or deep learning algorithms.

The technique of the invention provides high-resolution, high-contrast low-dose imaging at hard X-rays and/or gamma-rays, which may be further optimized by tailoring the mask pattern to a particular class of objects (regions of interest) or a specific object within a certain class, designing the compressed sensing algorithm and reconstruction algorithm in accordance with both mask pattern and object.

Detector(s) used to collect/detect radiation responses of the region of interest to the differently encoded X-ray beams include single-pixel detector or an array of single-pixel detectors or a flat panel.

The method for high-resolution, high-contrast low-dose imaging at hard X-rays and/or gamma-rays may further comprise digital image processing algorithms (e.g. denoising or edge enhancement), as well as automatic target recognition, classification and diagnosis.

The present disclosure also comprises a method for designing the masks/diffusers of the present disclosure. The method comprises planning the mask with both sufficient randomness and spatial thickness variance. Here, an algorithm, which provides the optimal results (that is best image quality with lowest required dose) is used; then the sparsity nature of the objects, for example, for various organs in the human body, is used to determine the reduction of the dose and the number of realizations, by using GI simulations combined with compressed sensing and/or machine learning (ML) tools. The structure of the mask/diffuser of the present disclosure may be further optimized by obtaining the cost function, which is the number of required realizations for obtaining the object's image with the best SNR. In order to obtain the cost function, ML tools, such as Support Vector Machine and deep learning using Artificial Neural Networks, may be used.

It should be noted that the present invention provides automatic classification of the regions of interest based on the results of the inspection (reconstructed image of the region of interest). The invention can advantageously be used in computed tomography, and in particular medical imaging, fluoroscopy, microscopy, non-destructive measurements.

According to one broad aspect of the invention, there is provided a masking assembly for use in inspecting a region of interest by creating structured high photon energy radiation to interact with the region of interest. The masking assembly comprises: at least one mask structure, the mask structure having a predetermined effective thickness t, and comprising a mask pattern formed by an arrangement of spaced-apart features of absorption properties with respect to said high photon energy radiation different from spaces between said features, said arrangement of said features along a lateral dimension of the pattern defining a predetermined effective pattern size P, wherein the features have a characteristic lateral size/providing a substantially high aspect ratio t/l.

In some embodiments, the mask structure has a spatial modulation of thickness along the pattern within said effective thickness t.

The mask structure may be a disk-like structure, where the spaced-apart features of the mask pattern are arranged along a substantially circular path.

The mask assembly may further include a drive mechanism configured and controllably operable as a displacement controller to implement successive lateral movements of said mask pattern with a predetermined lateral jump j to thereby enable successive inspection realizations through different segments of the mask pattern, respectively, wherein a value of said lateral jump j≠N×P. N being an integer. For example, the predetermined lateral jump j is smaller than P.

The aspect ratio t/l is at least 10, and preferably 10 or higher.

In some embodiments, the mask pattern is formed by relatively highly absorbing features arranged in the spaced-apart relationship in a relatively low-absorbing substrate with respect to said high photon energy radiation. In some other embodiments, the mask pattern is formed by relatively low-absorbing features arranged in the spaced-apart relationship in a relatively highly absorbing substrate with respect to said high photon energy radiation. The relatively high absorbing material of the mask pattern may include metal material composition or polymer-based material compositions.

Considering the metal material composition, this may include one or more of the following: tungsten, platinum, silver, gold and tantalum.

The mask pattern may be produced by metal printing of the spaced-apart features, followed by laser milling.

In some embodiments, the mask pattern is in the form of distribution of pores in the relatively highly absorbing substrate. Such substrate may for example be made from one or more of the following: Limestone, Mortar, AlCu, MgZn.

In some embodiments, the mask pattern comprises an arrangement of the features providing desired sparsity in a predetermined base selected in accordance with base of sparsity in the region of interest being inspected. For example, the arrangement of said spaced-apart features of the mask pattern is selected in accordance with a certain function, e.g., function corresponding to discrete cosine transform or the like, e.g. wavelet.

In some other embodiments, the mask pattern comprises a random arrangement of said spaced-apart features.

According to another broad aspect of the invention, it provides an inspection system for inspecting a region of interest, the inspection system comprising: a measurement device configured and operable to perform a plurality of successive measurements of the region of interest by implementing a corresponding plurality of interactions of the region of interest with high photon energy radiation beams having predetermined different spatially encoded intensity profiles, and a detection system comprising at least one radiation detector device successively collecting radiation responses of the region of interest to said interactions and generating measured data indicative thereof, wherein said high photon energy radiation beams having predetermined different spatially encoded intensity profiles are sequentially produced by passing an initial high photon energy radiation beam through the above-described masking assembly.

The at least one radiation detector device is configured and operable to collect said response of the region of interest comprising Compton scattering of the region of interest. The at least one radiation detector device may be accommodated for collecting at least one of back propagating and forward propagating radiation of said Compton scattering of the region of interest.

The inspection system also includes or is connectable to a control system configured and operable to process the measured data, thereby enabling reconstruction of image of the region of interest from said measured data by applying thereto at least one of compressed sensing based correlation processing and/or machine learning based processing, and generate data indicative of reconstructed image of the region of interest.

According to yet further broad aspect of the invention, it provides an inspection system for inspecting a region of interest, the inspection system comprising:
a measurement device configured and operable to perform a plurality of successive measurements of the region of interest by implementing a corresponding plurality of interactions of the region of interest with high photon energy radiation beams of X- or gamma-radiation having predetermined different spatially encoded intensity profiles, and a detection system comprising at least one radiation detector device configured and operable to successively collect radiation responses of the region of interest to said interactions and generate measured data indicative thereof, wherein said at least one radiation detector device is configured and operable to collect said response of the region of interest comprising Compton scattering of the region of interest including at least one of back propagating and forward propagating radiation of said Compton scattering of the region of interest, thereby enabling reconstruction of image of the region of interest from said measured data by applying thereto at least one of compressed sensing based correlation processing and machine learning based processing.

The inspection system may include a communication utility for communicating said measured data to a control system configured to perform said at least one of the compressed sensing based correlation processing and the machine learning based processing of the measure data and generate the reconstructed image of the region of interest. Alternatively or additionally, the inspection system includes a control system configured to perform said at least one of the compressed sensing based correlation processing and the machine learning based processing.

The measurement device comprises: a source of the X-ray or gamma-ray radiation configured to provide an input beam of said radiation; and a mask assembly accommodated in a general propagation path of said input beam, the mask assembly comprising at least one mask structure being configured and controllably operated to sequentially induce different intensity fluctuations on the input beam, thereby sequentially producing beams of the X- or gamma-radiation having predetermined different spatially encoded intensity profiles. The mask structure has a predetermined effective thickness t, and comprises a mask pattern formed by an arrangement of spaced-apart features of absorption properties with respect to said high photon energy radiation different from spaces between them, said arrangement of said features along a lateral dimension of the mask pattern defining a predetermined effective pattern size P, wherein the features have a characteristic lateral size l providing a substantially high aspect ratio t/l.

The inspection system further includes a drive mechanism configured and controllably operable to implement successive lateral displacements between the general propagation path of the input beam and said mask pattern with a predetermined lateral jump j to thereby enable successive inspection realizations through different segments of the mask pattern, respectively, wherein a value of said lateral jump j≠N×P. N being an integer.

The invention also provides a method for inspecting a region of interest by X-rays or gamma rays radiation comprising:
i) producing an input beam of X-ray or gamma-ray radiation directed along a general propagation path towards the region of interest;
ii) scanning, by the input beam on its way towards the region of inters, a mask pattern, thereby sequentially inducing different intensity fluctuations in the input beam and producing a sequence of beams of the X- or gamma-radiation having predetermined different spatially encoded intensity profiles propagating towards the region of interest to successively interact with the region of interest and induce radiation a corresponding sequence of radiation responses of the region of interest comprising Compton scattering of the region of interest, said scanning being implemented by successive lateral displacements between the general propagation path of the input beam and said mask pattern with a predetermined lateral jump j of a value satisfying a condition j≠N×P, where N is an integer and P is a predetermined effective pattern size P of the mask pattern;
iii) detecting said sequence of the radiation responses comprising at least one of back propagating and forward propagating radiation of said Compton scattering of the region of interest and generating measured data indicative thereof, thereby enabling reconstruction of image of the region of interest from said measured data by applying thereto at least one of the compressed sensing based correlation processing and the machine learning based processing.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 illustrates, by way of a block diagram, the inspection system according to the principles of the present invention;

FIG. 2 illustrates schematically the principles of configuring the mask pattern according to the present invention;

FIG. 3A illustrates an example of two step GI-based inspection technique of the invention;

FIG. 3B shows a specific example of a computer-designed mask suitable for use in the present invention; and FIG. 3C shows a photo of said mask;

FIG. 3D exemplifies a specific configuration of the system of the invention utilizing the two single-pixel detectors collecting backward scattered radiation response of a region of interest to spatially encoded radiation;

FIG. 4 shows the mask used for simulations and its various realizations;

FIGS. 5A to 5F show the process and results of simulations where the original image (FIG. 5A) was reconstructed with various auto-correlation lengths (ACL) and jumps;

FIG. 6 shows schematically machine learning based image reconstruction suitable to be used in the invention;

FIGS. 7A to 7C show simulations of resolution improvement for chest X-ray, wherein FIG. 7A shows a high-resolution image taken from images' bank; FIG. 7B shows the same image as FIG. 7A with artificially downgraded resolution; and FIG. 7C shows the simulated image using GI-based reconstruction scheme;

FIGS. 8A to 8B show simulations of compression for orthopedic images, wherein FIG. 8A shows a high-resolution image taken from images' bank; and FIG. 8B shows the result of GI simulation using compression ratio of 30 and a discrete cosine transform (DCT) basis for reconstruction; and

FIGS. 9A to 9B illustrate imaging of the Hebrew letter ‘Aleph’ (ϰ), wherein FIG. 9A shows experimental results of direct imaging; and FIG. 9B shows results of imaging using GI with about 500 realizations for the reconstruction of the 5000-pixel image.

### DETAILED DESCRIPTION OF EMBODIMENTS

The present disclosure encompasses systems and methods for high photon energies imaging. The present disclosure further comprises use of the systems and methods of the present disclosure for dose reduction in medical and non-destructive imaging, for resolution improvement, and for reducing scattering effects or for a single side operation in X-ray and gamma-ray measurements.

The present disclosure addresses the challenges associated with X-ray and gamma-ray imaging described above by utilizing a new approach that relies on: (1) Manipulating the structure of the beam that interacts with the object while employing advanced computational models; (2) Using correlation measurements; and (3) Combining the first two approaches with ML approaches.

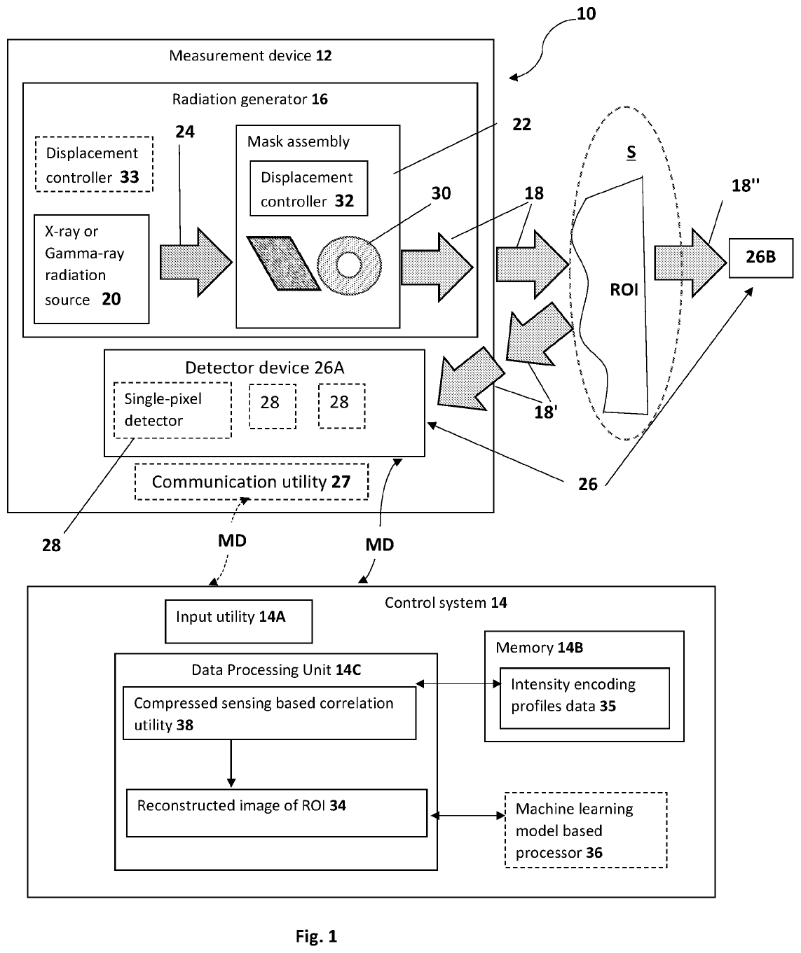

Reference is made to FIG. 1 illustrating, by way of a block diagram, an inspection system 10 configured and operable according to the principles of the present invention for inspecting a region of interest in a sample S (subject/object) and determining data indicative of a spatial structure of the region of interest, e.g. carrying information about defects/abnormalities of the region of interest. The system 10 is configured and operable to implement general principles of X- or gamma-ray based GI utilizing a sequence of interactions between the region of interest with different structured radiations, respectively.

The system of the invention can be used in various applications, including medical and other applications, such as computed tomography, fluoroscopy, microscopy, as well as non-destructive measurements. The latter can be used in inspection of samples for defects and other abnormalities.

The system 10 includes a measurement device 12 and is associated with a control system 14, i.e., includes or is connectable with the control system. As shown in the figure, the measurement device may include a communication utility 27 having any known suitable configuration for wireless data communication with the control system.

The measurement device 12 is configured and operable to perform a plurality of successive measurements of the region of interest by implementing a corresponding plurality of interactions of the region of interest with X-ray or gamma-ray radiation beams having predetermined different spatially encoded intensity profiles. In the description below, such radiation is referred to as X-ray radiation.

The measurement device 12 includes a radiation generator 16 configured and operable to sequentially produce such beams of X-ray or gamma-ray radiation having predetermined different spatially encoded intensity profiles 18 propagating towards a region of interest ROI; and a detection system 26 including at least one detector device 26A and/or 26B (each including one or more detectors 28 being preferably single-pixel detector(s)) configured and operable to collect successively induced responses of the region of interest to the interactions with said X-ray or gamma-ray radiation beams having predetermined different spatially encoded intensity profiles and generate measured data MD indicative thereof. The response of the region of interest being detected includes Compton scattering of the region of interest. The detection system 26 may include the detector device 26A accommodated to detect radiation response 18′ corresponding to back scattering of the spatially encoded radiation 18 from the region of interest and/or to detect radiation response corresponding to transmission 18″ of said radiation 18 through the region of interest.

The inventors have shown that collecting radiation 18′ back scattered from the sample might be sufficient to obtain all required information about the spatial structure of the sample, while enabling the inspection system of simpler configuration, smaller in footprint and weight. Also, the inventors have shown that the inspection results may be further optimized by detecting the back scattered radiation 18′ propagating along a path with as small as possible angular orientation with respect to the general propagation path of radiation 18.

The detector 28 may be a single pixel detector. The detector can use angular and/or spectral information. It should be understood that with the use of measured data including a sequence of measured data pieces indicative of a corresponding sequence of the radiation responses embedding different spatially encoded M intensity patterns, image of the region of interest can be reconstructed from the measured data with certain spatial resolution p×q (as if there were a matrix of p×q pixels), where p×q=N. N»M. To this end, compressed sensing based image processing is typically used, utilizing correlation between the M measured data pieces and prior knowledge of respective image data about the M mask pattern segments used in the respective realizations.

The present invention provides ways for implementing such technique with a reduced number M of realizations/acquisitions, and thus lower dose of radiation applied to the region of interest, and potentially shorter inspection session, as well as simpler, smaller and lighter mask. This is achieved by an optimized configuration of the mask structure and its displacement to provide the sequence of beam interactions with different mask pattern segments. This will be described more specifically further below.

The radiation generator 16 includes a radiation source 20 producing a beam of X-ray or gamma-ray radiation 24 (of a predetermined spectral range). The radiation generator further includes a mask assembly/unit 22 which includes at least one mask defining a mask pattern formed by an arrangement of spaced-apart features of absorption properties with respect to said radiation different than that of spaces between these features.

The radiation generator 16 is configured to provide controllable relative displacement between the mask pattern and a general propagation path of the input radiation beam 24 such that the beam 24 sequentially passes through different (mutually different) segments of the mask pattern thus resulting in a sequence if differently spatially encoded beams sequentially interacting with the region of interest.

As shown in the figure, in some embodiments, the mask is controllably laterally displaceable/movable with respect to the general propagation path of the radiation beam 24 to cause said beam to successively pass through different pattern segments of the mask pattern to thereby induce different spatial encoding in the intensity profile of the beam. To this end, the mask assembly includes a drive mechanism/displacement controller 32 configured and operable to control displacement (e.g., rotation of a disk-like pattern) with respect to the propagation path during an inspection session to implement a plurality of M different realization/acquisitions with M mutually different spatially encoded radiation distributions 18.

Alternatively, or additionally, to the mask pattern displacement with respect to the input beam propagation path, the input beam 24 propagation path can be controllably displaced with respect to the mask pattern using a respective displacement controller 33.

The displacement of the mask pattern with respect to the radiation propagation path is implemented with a predetermined lateral step/jump j the value of which is selected such that it is different from k×P, where k is an integer and P is a predetermined effective pattern size P of the mask pattern. The preferred embodiments for the mask pattern configuration are described further below.

The control system 14 is generally a computer system including inter alia such functional utilities as input utility 14A, memory 14B and data processor 14C. As indicated above, the control system 14 may be part of the measurement device 12 or may be a remote system in data communication with the measurement device (with the detection system) via a communication network. It should be noted that data processing modules may be distributed between a local controller of the measurement device and the remote control system, as the case may be.

The control system 14 is configured and operable to process the measured data MD to obtain reconstructed image of the region of interest 34. This can be implemented using a compressed sensing based correlation utility 38 configured and operable based on the general principles of compressed sensing based image processing utilizing correlation between the M measured data pieces and prior knowledge of respective image data about the M mask pattern segments, i.e. intensity encoding profiles data 35 stored in the memory 14B, used in the respective realizations. Alternatively, utilizing machine learning model based data processor 36 can be used.

The mask pattern includes an arrangement of spaced-apart features which differ from the spaces between them in the interaction properties with said radiation 24, e.g., absorption, transmission, scattering of said radiation. The mask pattern operates as a radiation diffuser and may be transversely/laterally displaceable with respect to the radiation propagation path (e.g. via rotation in a plane substantially perpendicular to the radiation propagation path) using a predetermined lateral displacement step/jump.

The pattern may include a periodic (or almost periodic) arrangement of features, or arrangement according to a certain known function. In some embodiments, the mask includes a random arrangement of features. Thus, passage of radiation 16 through the patterned segment of the mask results in the spatially encoded radiation 18 which interacts with the region of interest.

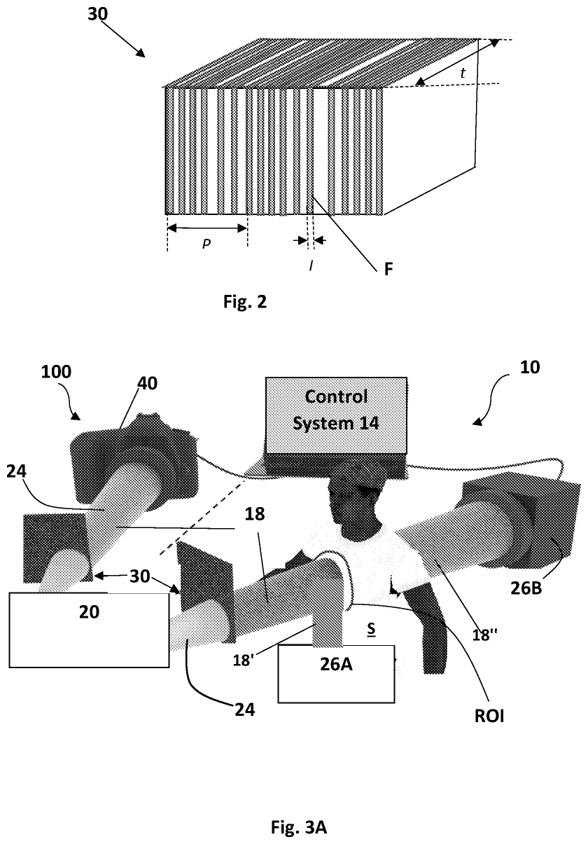

Reference is made to FIG. 2 schematically illustrating the main principles for configuring the mask structure 30 and the mask pattern. It should be understood that the figure is schematic, not in scale, and the pattern is simplified just in order to simplify the description of the main features thereof.

The mask structure 30 (e.g. plate-like structure) has a certain predetermined effective thickness t and is formed with a predetermined pattern of the arrangement of spaced-apart features F which have different absorption properties (and thus transmission) with respect to the X-ray radiation used in the inspection procedure as compared to that of spaces between them. In some embodiments, the mask structure (e.g. plate-like structure) is configured with spatial modulation of thickness along the pattern within said effective thickness t.

For example, the features F are relatively highly absorbing (less transmissive) than the spaces between these features, or alternatively, features F are almost non-absorbing in highly-absorbing bulk of the mask structure. It should also be noted that the pattern may be such that the features F as well as spaces between them, although being relatively transmissive and absorbing, the features F may be different between them in the respective property, and similarly, the spaces nay be different between them in their respective properties.

Thus, generally speaking, the mask pattern is in the form of varying transmission with respect to said X-ray radiation, where either highly absorbing features are arranged in the spaced-apart relationship in a relatively low-absorbing substrate with respect to said high photon energy radiation, or relatively low-absorbing features are arranged in the spaced-apart relationship in a relatively highly absorbing substrate. The relatively high absorbing material of the mask pattern may include metal material composition(s), e.g. one or more of the following: tungsten, platinum, silver, gold and tantalum.

The mask pattern may be produced by metal printing of the spaced-apart features, followed by laser milling, in the low-absorbing substrate of the mask structure. Alternatively, the mask pattern may be in the form of distribution of pores in the relatively highly absorbing substrate.

For example, the arrangement of features may be random or quasi-random. In some embodiments, the mask structure (plate-like structure) is configured with both the spatial modulation of thickness and the randomness of the pattern.

The feature F has a characteristic lateral size l, such that a substantially high aspect ratio t/l is achieved (e.g., the aspect ratio of at least 10). The pattern (arrangement of features F) has its predetermined effective pattern size P. Such pattern size P presents a so-called “correlation length” of the mask, which describes an “effective pixel size”, corresponding to the number of adjacent pixels of the same value in the assumed pixel matrix p×q of the required spatial resolution of the reconstructed image. This is actually an autocorrelation characteristic of the mask pattern.

Further, according to the invention, the displacement controller 32 is configured and operable to provide optimized controllable lateral displacement jump j of the mask/pattern such that j≠k≠P, k being an integer. More specifically, the jump j may be smaller or larger than P but should not be of the value of multiplication of P.

In some embodiments, the mask pattern is such that the arrangement of features F provides desired sparsity in a predetermined base, which is selected in accordance with the base of sparsity in the region of interest. To this end, a preliminary imaging may be performed once for a given region of interest using the same X-ray radiation with no spatial encoding of such radiation (no mask) and using a pixelated detector, and analyze the image data to identify the base of sparsity of the region of interest.

With some prior knowledge regarding the type and size of the imaged object, this can be made even without the preliminary imaging stage.

In some other embodiments, the arrangement of features F in the mask pattern is selected in accordance with a certain function, e.g. a function corresponding to discrete cosine transform. This will be described further below.

The jump-by-jump lateral displacement of the mask pattern with respect to the general propagation path of X-ray radiation results in successive location of different pattern segments in the propagation path of the radiation thus differently encoding such radiation. The mask patterns (or pattern segments of the mask) may be a-priori known (predetermined, e.g. according to a certain function) and fabricated based on a computer-generated predesigned map. Alternatively, the patterns may be unknown and previously (prior to inspection) characterized using a high-resolution system. To this end, the spatial intensity profiles of the X-ray radiation induced by the pattern segments can be previously studied by interacting the X-ray radiation with each pattern segment, detecting the response of the pattern segment (transmission or reflection) by a pixelated detector, and storing the respective image data. Such technique is generally known and used in GI applications.

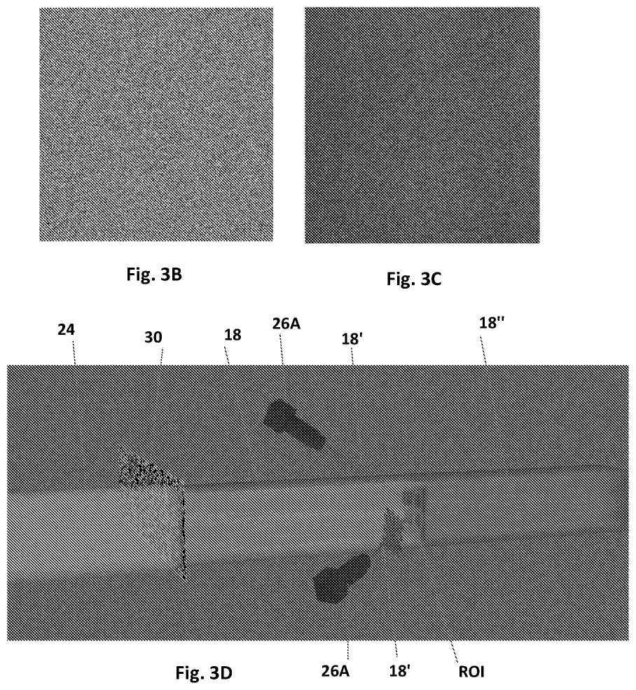

Reference is made to FIG. 3A schematically exemplifying the configuration and operation of the inspection system 10 of the invention. In this example, the system 10 includes a standard X-ray source 20 typically used for medical imaging. As shown in the fire, the pattern segments of the mask 30 is first learned using an imaging system 100, which utilizes the same X-ray source 20 and a pixelated detector 40. It should be understood that the mask is laterally displaced (e.g. rotated) and the input beam 24 thus successively interacts with (passes through) a different pattern segment and is then detected by the pixelated detector 40. It should be understood that the imaging plane (radiation sensitive surface of the pixelated detector) is the same or conjugate plane of the detector 26A (or 26B) which is then used in the inspection of the region of interest.

The mask 30 may be fabricated by either nanotechnology or 3D printing, where two materials (e.g. stainless steel and a polymer) may be used to achieve the high absorption contrast that introduces the structured illumination. According to said procedures, the patterns of the mask are predesigned, using a computer code, for example, by using MATLAB, and the fabrication of the mask is performed according to the design. For example, this may be a computer-generated mask. According to this procedure, the landscape of the mask is known and is used to calculate the intensity fluctuations at the object position.

In the experiments conducted by the inventors, the structure of the mask was predesigned with a MATLAB code, hence the structure of the fluctuation intensity was known in the entire range of realizations. FIGS. 3B and 3C illustrate the designed mask and a photo of the mask. The mask 30 shown in FIG. 3B is designed to be used at 661 keV gamma-ray radiation. The average lateral feature size is about 110 μm and its height (mask plate thickness) is about 1.5 mm. Therefore, correlation length (effective pattern size) is also about 110 μm and the spatial resolution of the mask image is comparable.

During the inspection of the region of interest, the mask is preferably positioned as close as possible to the object (containing the region of interest) in order to avoid magnification of the feature size of the mask in the image plane (defined by the single pixel detector). The mask pattern is scanned (i.e. via lateral displacement of the mask in one or two lateral dimensions), and the successive differently encoded responses of the region of interest are detected by the single-pixel detector 26A (and/or 26B) and the corresponding successive measured data pieces (forming the measured data) are recorded (stored). Then, this measured data is processed and the image of the region of interest is reconstructed. This may be done using any known suitable technique based on correlation of the radiation intensity measured by the single-pixel detector and the intensity profile of the mask pattern (pattern segment) for each of the realizations.

For lower photon energies of X-ray radiation, masks with smaller features are used, thereby improving the spatial resolution. This is possible since the absorption of materials is larger at lower photon energies and thus the intensity induced fluctuations by a thin mask are more significant in this range. Since the mechanical constraints limit the ratio between the thickness of the feature and its lateral dimensions, the low absorption at high photon energies leads to the requirement of millimeter scale thicknesses, and therefore the lateral dimensions are about 100 microns. However, at lower photon energies it is possible to use even thicknesses on the order of 10 microns. For example, in mammography the typical photon energy is between 20 keV and 30 keV. In this range masks with feature sizes down to 10 microns, which are made either from porous materials or that are made by photolithography, are used. For the porous materials, their pattern is to be measured before the measurement/inspection of the object, and the two-step approach described above with reference to FIG. 3A is used. The mask is therefore positioned to modulate the intensity of the input beam 24 in the absence of the object, and the patterns of the masks (pattern segments of the same mask) are imaged for the various realizations that are used in the second step where the object is measured/inspected. In the second step, the object (region of interest) to be inspected is located in the path of the X-ray radiation 24 and the intensity is measured by the single-pixel detector(s) as described above. As with the known mask, the computational approach described above is used to reconstruct the image of the object (region of interest).

For the intermediate energy range of 30 keV-100 keV, which is routinely used for various medical examinations, mask patterns with feature sizes of about 20-50 microns can be used, improving thereby the state-of-the-art medical imaging resolution. As described above, the constraint is the ratio between the thickness and lateral dimensions of the features (aspect ratio t/l), which depends on the details of the manufacturing technique, and is at least 10, i.e. at least one order of magnitude higher, and preferably 3 or more orders of magnitude higher. There are many suitable known techniques for the fabrication of the mask, for example three-dimensional metal printing and laser milling of heavy elements (e.g., tungsten or platinum).

When using the Compton scattering, which is the dominant scattering mechanism in the X-ray regime, the procedure for the image reconstruction may be generally similar to the above-described compressed sensing and correlation based technique, but the detector is to be positioned to collect the Compton scattered signal (detector 26A) instead of or in addition to the transmitted signal (detector 26B). Since the single-pixel detector has no spatial resolution, it is not sensitive to the angle of the scattered radiation in contrast to standard pixelated detectors.

The inventors have shown that using the technique of the invention utilizing detection of the Compton effect scattering with the single-pixel detector(s) allows to reconstruct the image of the region of interest even when the source and the detector are on the same side of the object (i.e. backward propagating response of the region of interest).

The schematic of the experimental setup is depicted in FIG. 3D in a self-explanatory manner where two such single-pixel detectors 26A are used to collect backward propagating responses of the region of interest substantially symmetrically with respect to the general propagation path.

As indicated above, the mask pattern (arrangement features of different properties with respect to the X-ray radiation) can be designed in accordance with a predetermined function. The inventors have shown that the function corresponding to a discrete cosine transform (DCT) can be used in the above described configuration of the mask. The following is the theoretical explanation supported by simulations conducted by the inventors.

In ghost imaging a mask/diffuser (D) is used to illuminate an object (\vec{x}) and accumulate all the transmitted (or reflected/scattered) radiation into a bucket detector (\vec{b}) (e.g., a single-pixel detector) a number of times (M realizations). For each realization, the mask/diffuser D is moved so that each successive realization utilizes a different pattern segment and is different from the preceding one, and at each realization a virtual matrix p×q of pixels of the respective pattern segment of the mask/diffuser is exposed to the process. By measuring the transmission (or reflection) of diffuser D, the object \vec{x} (with resolution of p×q pixels) may be reconstructed via the bucket readings \vec{b}.

The following equation describes the measurement process using here, for simplicity, a one-dimensional problem (in the case of a two-dimensional object whose size is [p×q] pixels, the total number of pixels, N, is N=p×q):

Dx=b (1)

where D is an M×N matrix of the diffuser/mask pattern whose rows are the M different realizations (mask transmission/scattering realizations) for each pixel in the virtual matrix p×q, \vec{x} is the N (unknown) object transmission/scattering values for each pixel and \vec{b} is the vector of bucket detector measurements (e.g., single-pixel detector) for each mask realization. Mask realizations could be obtained using the same physical mask translated or rotated in front of the source. Each mask position corresponds to one measurement/realization.

Using compressed sensing, it is possible to obtain more information with fewer (i.e., smaller number M) of measurements by treating the process as a constraint of linear equations (size of M×p×q) and trying to minimize various possible norms.

In the following, the inventors show that with a certain diffuser/mask pattern, the norm of a gradient (as well as the norm of higher derivatives) is already minimal. Therefore, here lies a degree of freedom to choose a different norm to minimize or to satisfy other constraints.

In general, ghost imaging (GI) uses the following set of linear equations (respectively, for one-dimensional (1D) and two-dimensional (2D) problem):

$$\{ \begin{matrix}D \vec{x} = \vec{b} & 1 D \\ {\sum }_{j , k} {D}_{ijk} {x}_{jk} = {b}_{i} & 2 D\end{matrix} \qquad ( 2 )$$

The number of measurements is the number of rows in D (denoted by i), and hence, if there are enough measurements, then D is invertible. To try and minimize the number of measurements (and therefore the amount of radiation) the inventors employed various compressed sensing techniques.

In the Total Variation Algorithm 3 (TVAL3) one tries to minimize Eq. (3)

$$ℒ = \{ \begin{matrix}α {\vert D \vec{x} - \vec{b} \vert}_{2} + β {\vert ∇ x \vert}_{1} & 1 D \\ α {\vert D \vec{x} - \vec{b} \vert}_{2} + β ( {\vert {\partial }_{x} x \vert}_{1} + {\vert {\partial }_{y} x \vert}_{1} ) & 2 D\end{matrix} \qquad ( 3 )$$

or, in an alternative notation:

$$ℒ = \{ \begin{matrix}α \sqrt{{\sum }_{i} {( {\sum }_{j} {D}_{ij} {x}_{j} - {b}_{i} )}^{2}} + β {\sum }_{j} \vert {x}_{j + 1} - {x}_{j} \vert & 1 D \\ \begin{matrix}α \sqrt{{\sum }_{i} {( {\sum }_{j , k} {D}_{ijk} {x}_{jk} - {b}_{i} )}^{2}} + \\ β {\sum }_{j , k} ( \vert {x}_{j + 1 , k} - {x}_{j , k} \vert + \vert {x}_{j , k + 1} - {x}_{j , k} \vert )\end{matrix} & 2 D\end{matrix} \qquad ( 4 )$$

If α»β (which usually is, otherwise one would obtain a flat line where the gradient is zero), then the corresponding constraint is more important. It can be seen that if the diffuser rows are orthogonal the object can be reconstructed using these vectors minimizing |D\vec{x}−b|₂=0 (or |D\vec{x}−b|₂˜0 if α [symbol] β) and to uphold that constraint one is allowed only to change the orthogonal complements (or very little of the diffuser vectors so that |D\vec{x}−b|₂˜0).

The next step is to transform the diffuser matrix and the object vector to the basis in which the object is sparse. The transform which is used here for the analysis is the discrete cosine transform (DCT). However, this could be any other transform which satisfies the conditions of compressive sensing which are: (1) that the signal has a sparse representation in this basis; and (2) that the measurement basis is incoherent with the basis in which the signal is sparse.

The discrete cosine transform (DCT) algorithm is commonly used for image compression. It converts the pixels in an image into sets of spatial frequencies and is closely related to the DFT (Discrete Fourier Transform). Each realization is a basis vector of the discrete cosine transform (DCT), as will be explained in detail in the following. The discrete cosine transform (DCT) in 1D is defined as:

$$C [ l ] = \sqrt{\frac{2}{q}} {\sum }_{i = 0}^{q - 1} c [ k ] cos ( \frac{π}{q} ( k + \frac{1}{2} ) l ) \qquad ( 5 )$$

where c[k] is the k^{th} pixel of object c, and C[l] is the l^{th} pixel of the DCT (counting from 0 to q−1). To get the transformation matrix and how it looks in real space one needs to apply the inverse DCT (IDCT) to each row of the identity matrix. Each row of the identity is a basis vector. Thus, essentially, it amounts to just transforming the basis vectors. Then each row in the transformation matrix will represent a DCT basis vector. The IDCT is:

$$c [ k ] = \sqrt{\frac{1}{q}} C [ 0 ] + \sqrt{\frac{2}{q}} {\sum }_{l = 1}^{q - 1} C [ l ] cos ( \frac{π}{q} ( k + \frac{1}{2} ) l ) \qquad ( 6 )$$

For the i^{th} vector the following is obtained:

$${c}_{i} [ k ] = \sqrt{\frac{1}{q}} {C}_{i} [ 0 ] + \sqrt{\frac{2}{q}} {\sum }_{l = 1}^{q - 1} {C}_{i} [ l ] cos ( \frac{π}{q} ( k + \frac{1}{2} ) l ) \qquad ( 7 )$$

Because the identity matrix is transformed: Cᵢ[l]=δᵢₗ

$${c}_{0} [ k ] = \sqrt{\frac{1}{q}} {δ}_{i 0} + \sqrt{\frac{2}{q}} {\sum }_{l = 1}^{q - 1} {δ}_{l 0} cos ( \frac{π}{q} ( k + \frac{1}{2} ) l ) = \sqrt{\frac{1}{q}} \\ {c}_{i \neq 0} [ k ] = \sqrt{\frac{1}{q}} {δ}_{i 0} + \sqrt{\frac{2}{q}} {\sum }_{l = 0}^{q - 1} {δ}_{il} cos ( \frac{π}{q} ( k + \frac{1}{2} ) l ) = \sqrt{\frac{2}{q}} cos ( \frac{π}{q} ( k + \frac{1}{2} ) i ) \qquad ( 8 )$$

Since {Cᵢ[l]}ᵢ₌₀^{q-1} are orthonormal and the transformation is unitary, it imposes that {cᵢ^{[k]}}ᵢ₌₀^{q-1} are also orthonormal (Because the inner product doesn't change under the transformation). Meaning Σₖ₌₀^{q-1}cᵢ[k]cⱼ[k]=δᵢⱼ.

The inventors use {cᵢ[k]}ₒ₌₀^{M} as the form for the DCT based diffuser with M realizations. The compact form can be introduced:

$${c}_{i} [ k ] = {a}_{i} cos ( {ω}_{i} k + {ϕ}_{i} ) \qquad ( 9 )$$

where

$${a}_{i} = \sqrt{\frac{2 - {δ}_{i 0}}{q}}$$

includes the normalization constant,

$${ω}_{i} = \frac{π i}{q}$$

is the frequency,

$${ϕ}_{i} = \frac{π i}{2 q}$$

is the phase and k is the k^{th} pixel.

Following the previous logic, the next picture/image (i.e., required pixels of the object) in 1D (suggesting p=1, but it can be naturally extended to 2D) is as follows:

$$x ( k ) = {\sum }_{i = 0}^{q - 1} {c}_{i} [ k ] = {\sum }_{i = 0}^{q - 1} {a}_{i} cos ( {ω}_{i} k + {ϕ}_{i} ) \qquad ( 10 )$$

Now, taking a gradient with respect to k one obtains

$$\begin{matrix}x ( k + 1 ) - x ( k ) = {\underbrace{\frac{\partial }{\partial k} x ( k )}}_{in the continuum limit} = - {\sum }_{i = 0}^{q - 1} {ω}_{i} {a}_{i} sin ( {ω}_{i} k + {ϕ}_{i} ) = \\ = {\sum }_{i = 0}^{q - 1} {\tilde{a}}_{i} sin ( {ω}_{i} k + {ϕ}_{i} )\end{matrix} \qquad ( 11 )$$

which again consists of orthogonal basis elements:

$$( {\sum }_{k = 0}^{q - 1} {\tilde{a}}_{i} sin ( {ω}_{i} k ) {\tilde{a}}_{j} sin ( {ω}_{j} k ) = {δ}_{ij} ) .$$

Eq. (5) was obtained by the simulation and it is a constraint. Thus, any other additions of basis elements will only raise

$${\vert \frac{\partial }{\partial k} x ( k ) \vert}_{1} .$$

Because if {\vec{xₗ}} is a set of orthogonal vectors and a vector is represented as \vec{v}=Σᵢ₌₀^{p}αᵢ\vec{xₗ}, an addition of another basis element will change the vector to \vec{w}=Σᵢ₌₀^{p+1}αᵢ\vec{xₗ} (aₚ₊₁≠0) and the norms will be:

$$\vert \vec{v} \vert = {\sum }_{i = 0}^{p} \vert {a}_{i} \vert < \vert \vec{w} \vert = {\sum }_{i = 0}^{p + 1} \vert {a}_{i} \vert = \vert \vec{v} \vert + \vert {a}_{p + 1} \vert$$

(the inequality is also valid for the L₂ norm). Here the gradient's norm is increased from the same considerations.

Thus, a DCT diffuser provides a minimal gradient and the results of the TVAL3 are similar to reconstruction via the sum of the basis vectors. The DCT diffuser reconstruction complexity is M×p×q whilst the complexity of TVAL3 is not deterministic and can greatly exceed this complexity as a single iteration requires more than (M×(m×n−1))² operations because of matrix multiplication.

In the following, the inventors simulated a realistic random mask/diffuser with a certain effective pattern size, i.e., autocorrelation length (ACL), and investigated how the ACL and the jumps over the diffuser/mask pattern influence the reconstruction of an object (region of interest). In particular, the inventors tested the effect of overlaps of pattern portions between different realizations of one mask pattern on the quality of reconstruction. The motivation for creating a quality reconstruction based on high-overlap realizations is the possibility of shortening the measurement time as well as reducing the physical size of the mask. Shortening of the measurement time may result from using smaller movements/jumps of the mask, while keeping the same number of realizations, whereas smaller movements may also contribute to reduction of the overall size of the mask.

The mask patterns designed for the simulations are random patterns based on a normal distribution, since this is a relatively easy format to produce in practice.

The simulations differ from each other in three parameters:

1. The compression factor, which determines the number of different realizations M, defined by:

$$M = ⌊ \frac{p \times q}{compression factor} ⌋$$

2. The length of the mask auto-correlation. It should be kept in mind that the term “auto-correlation length” (ACL) as used here does not refer to auto-correlation in the mathematical-statistical meaning, but to the number of adjacent pixels (i.e., under assumed resolution of p×q pixels) whose value is the same. In other words, the ACL as used here expresses the degree to which the distribution of lateral sizes of the spaced-apart features F of the mask is self-similar laterally on the mask. The respective area of adjacent pixels whose value is the same is henceforward referred to as “effective pixel”.

3. The size of the overlap between the realizations, which is determined by the size/value of the displacement (jump) on the mask pattern between any two realizations. Depending on the lateral axis, the jump may be referred to as jumpₓ or jump_{y}, as in the example below.

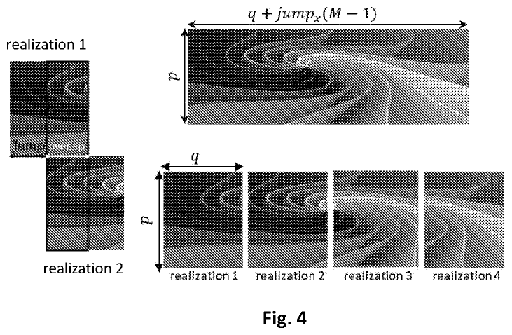

Referring to FIG. 4, an exemplary mask pattern is shown and the pattern segments corresponding to four realizations based on the mask displacements (“jumpₓ”) with respect to the X-axis. As shown in the figure, the pattern segments are designed so that their size is affected by combining two of the three parameters: [p×q+jumpₓ×(M−1)]

Each pattern segment is simulated by:
(i) randomization of an

$$⌈ \frac{p}{ACL} ⌉ \times ⌈ \frac{q + {jump}_{x} \times ( M - 1 )}{ACL} ⌉$$

array from a normal distribution with mean=0.5, variance=0.3;
(ii) limiting the mask transfer (i.e., absorption) values to be between 0 to 1 in order to simulate absorption values and thus creating a difference between one realization and the other; and
(iii) duplicating each pixel to create a uniform area with a size of ACL×ACL—an “effective pixel”.
   This above-defined matrix D simulates a sandpaper-like mask pattern. Simulations of several types were performed: one-dimensional objects (region of interest); and two-dimensional objects when the overlap between different realizations is in the X-axis only.

In order to simplify the simulation, there is no reference to the progress of the field in the space between the pattern segment and the single-pixel detector, and it is assumed that this is the same field at both points, and the field can be linked to a system where the detector is attached. In addition, in the first stage, no-noise simulations were performed, simulating a simple system in which the following two conditions are met:

1. A uniform beam without source noise—the radiation from the source has a constant intensity in space (as opposed to e.g., a Gaussian beam) and in addition the source is uniform without noise, both in time and in space.

2. A pattern segment with perfect effective pixels so that the value of each effective pixel is uniform.

In practice, the two conditions above are not met in the physical system, where the source noise impairs the quality of the reconstruction while the differences in transfer values within an effective pixel in the mask improve it.

To examine the effect of source noise on the quality of the reconstruction, several simulations were performed with the source noise added. In order to isolate the effect of the source noise which is expected to impair the quality of the restoration, no noise was introduced for this stage for the mask itself.

A number of quality parameters were calculated with the purpose of examining the quality of the reconstructions quantitatively: PSNR-peak signal-to-noise ratio, SSIM —structural similarity index measure, relative error, and mean squared error.

For a noise-free p×q, monochrome image K and a noisy reconstruction U, the PSNR (in dB) is defined as

$$PSNR = 10 \cdot {log}_{10} ( \frac{{MAX}_{K}^{2}}{MSE ( U , K )} ) \qquad ( 12 )$$

### The SSIM is:

$$SSIM ( U , K ) = {[ 1 ( U , K ) ]}^{α} \cdot {[ c ( U , K ) ]}^{β} \cdot {[ s ( U , K ) ]}^{γ} \qquad ( 13 )$$

$$where :$$

$$l ( U , K ) = \frac{2 {μ}_{U} {μ}_{K} + {c}_{1}}{{μ}_{U}^{2} + {μ}_{k}^{2} - {c}_{1}} ,$$

$$c ( U , K ) = \frac{2 {σ}_{U} {σ}_{K} + {C}_{2}}{{σ}_{U}^{2} + {σ}_{K}^{2} - {C}_{2}} ,$$

$$s ( U , K ) = \frac{{σ}_{UK} + {C}_{3}}{{σ}_{U} + {σ}_{K} + {C}_{3}} ,$$

α=β=γ=1, C₁=0.01 L, C₂=0.03 L,

$${C}_{3} = \frac{{C}_{2}}{2} ,$$

L=1 (the dynamic range of the pixel values), μᵢ, σ_{U}² are the mean and the variance of U respectively, and the same for K. For simplicity, movement in one direction (x direction) over the mask was assumed. The simulated mask pattern was chosen with an ACL of c pixels that moves with constant jumps of length j and undergoes M measurements of an object with a size of p×q pixels by drawing a

$$⌈ \frac{p}{c} ⌉ \times ⌈ \frac{q + j ( M - 1 )}{c} ⌉$$

normal random array (mean=0.5, variance=0.3) and replacing each element of the array with an array of the size c×c with the original element repeated inside. This creates a virtual pixel matrix of the mask pattern.

To simulate a realistic mask pattern, the absorption property of the different features of the pattern to the X-ray radiation is between 0 and 1. The above distribution was chosen in order to simulate a random, e.g., a sandpaper-like mask pattern However, many other kinds of distributions could have been chosen with similar results achieved (it will be especially apparent when the mask pattern is represented in a matrix form below).

A 2D object can be rearranged in a 1D form and thereby the discussion of the 1D case will still be valid.

The first investigation concerns the case of jumps which are integer multiples of the ACL (c). In the 1D (p=1) case a block matrix is obtained:

$$D = ( \begin{matrix}[ \begin{matrix}{a}_{00} & … & {a}_{00}\end{matrix} ] & … & [ \begin{matrix}{a}_{0 q} & … & {a}_{0 q}\end{matrix} ] \\ ⋮ & ⋱ & ⋮ \\ [ \begin{matrix}{a}_{N 0} & … & {a}_{N 0}\end{matrix} ] & … & [ \begin{matrix}{a}_{Nq} & … & {a}_{Nq}\end{matrix} ]\end{matrix} ) \qquad ( 14 )$$

The constraints are:

D\vec{x}=\vec{b} (15)

This can be rewritten as:

$$\tilde{D} \vec{χ} = ( \begin{matrix}{a}_{00} & … & {a}_{0 q} \\ ⋮ & ⋱ & ⋮ \\ {a}_{N 0} & … & {a}_{Nq}\end{matrix} ) \vec{χ} = \vec{β} \qquad ( 16 )$$

whilst \vec{β}=\vec{b}/c.
   A solution for (16) is also a solution for (15) in a sense that every element in χ is repeated in x for c (i.e., ACL) times.

$${x}_{i} = {χ}_{⌊ \frac{i}{c} ⌋} \qquad ( 17 )$$

For computational ghost imaging (CGI), the Total Variation Algorithm 3 (TVAL3) is used, which tries to minimize (18):

$$ℒ = α \sqrt{{\sum }_{i} {( {\sum }_{j} {D}_{ij} {x}_{j} - {b}_{i} )}^{2}} + γ {\sum }_{j} \vert {x}_{j + 1} - {x}_{j} \vert \qquad ( 18 )$$

with α»γ.
The solution in (17) fits perfectly to the reduction of the norm |∇x|₁=Σⱼ|xⱼ₊₁−xⱼ| because it reduces the average gradient. The larger c is, the more explicitly one expects to observe this behavior because the larger is the value of c, the lower is the average gradient.

Let us consider the dimensions of matrix \tilde{D}

$$( i . e . , M \times ⌈ \frac{q}{c} ⌉ ) .$$

The dimensions of D were M×q, with M<q, because of the compression. Now,

$$⌈ \frac{q}{c} ⌉ < q$$

and one might even have

$$⌈ \frac{q}{c} ⌉ < M .$$

So, these computations are valid under

$$M > ⌈ \frac{q}{c} ⌉ .$$

To reduce the gradient, \tilde{D}_{\vec{χ}}=\vec{β} is solved under

$$M > ⌈ \frac{q}{c} ⌉$$

employing TVAL3.

A convenient way to reorganize D is by periods, which means rearranging the rows so that each set of rows will have the same block structure as in (14). The higher the number of the sets, the less |∇x|₁ will cause a block structure as in (16) and the number of such sets is given by

$$\frac{ACL}{gcd ( ACL , Jump )} ( gcd = greatest common divider ) .$$

Table 1 below shows the fraction ACL/gcd(ACL,Jump) for several choices of the ACL and jump. Larger integers in the Table 1 correspond to a higher probability for a better reconstruction. These numbers indicate the trend expected in the simulation.

TABLE 1

| | | Jump | | | | | | | |
|---|---|---|---|---|---|---|---|---|---|
| | | 2 | 4 | 7 | 13 | 27 | 40 | 70 | 100 |
| ACL | 2 | 1 | 1 | 2 | 2 | 2 | 1 | 1 | 1 |
| | 4 | 2 | 1 | 4 | 4 | 4 | 1 | 2 | 1 |
| | 7 | 7 | 7 | 1 | 7 | 7 | 7 | 1 | 7 |
| | 10 | 5 | 5 | 10 | 10 | 10 | 1 | 1 | 1 |
| | 13 | 13 | 13 | 13 | 1 | 13 | 13 | 13 | 13 |
| | 20 | 10 | 5 | 20 | 20 | 20 | 1 | 2 | 1 |

Table 2 below shows the PSNR of the simulation under compression ratio (CR) of 20 times. To prove the consistency of the results, each value was obtained as a mean of 100 simulations. It can be seen that for the larger ACL values, theory and simulation coincide to a large extent.

TABLE 2

| | | Jump | | | | | | | |
|---|---|---|---|---|---|---|---|---|---|
| | | 2 | 4 | 7 | 13 | 27 | 40 | 70 | 100 |
| ACL | 2 | 15.34 | 17.38 | 18.30 | 18.39 | 18.15 | 17.94 | 18.12 | 18.06 |
| | 4 | 15.47 | 19.53 | 19.77 | 19.69 | 19.70 | 20.25 | 19.64 | 20.28 |
| | 7 | 17.20 | 21.66 | 20.41 | 22.17 | 22.16 | 22.21 | 20.40 | 22.16 |
| | 10 | 20.95 | 21.52 | 21.06 | 21.13 | 21.17 | 18.79 | 18.78 | 18.78 |
| | 13 | 20.56 | 20.68 | 20.71 | 17.65 | 20.75 | 20.76 | 20.75 | 20.75 |
| | 20 | 19.53 | 19.17 | 19.60 | 19.62 | 19.62 | 16.19 | 17.96 | 16.19 |

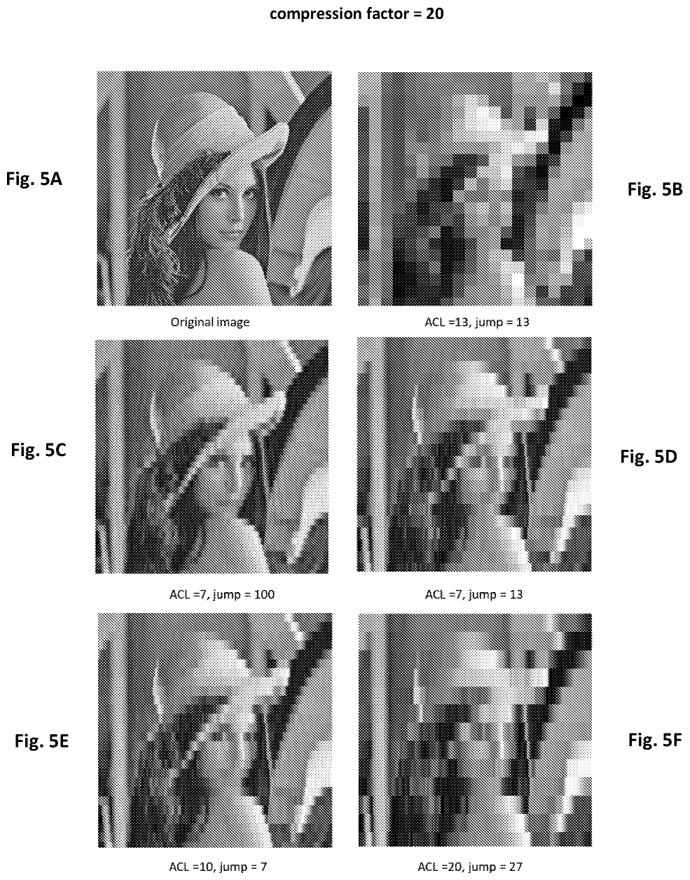

FIGS. 5B to 5F show examples of (simulated) reconstructions of an original image shown in FIG. 5A. As already mentioned above, all the mask patterns were derived from a normal distribution with μ=0.5 and σ=0.3.

The main simulation result demonstrates that an image of the region of interest can be better reconstructed with jumps that are smaller than the ACL (and consequently, the mask can be smaller). This can be seen, for example, comparing the PSNR values in rows corresponding to ACL=13 or ACL=20 in Table 2 and comparing the image quality of FIG. 5D (ACL=7, jump=13) and FIG. 5E (ACL=10, jump=7). Though, for an ACL=7 for example, Jump=13 is better than Jump=4 but a bigger jump, e.g., Jump=40 or Jump=100 pixels (refer to PSNR values in Table 2) does not seem to improve much the reconstruction quality (i.e. increase PSNR).

It is to be noted that the results of the simulation show that the jump (i.e., displacement step of mask pattern) is to be of an optimal size with respect to the ACL. Table 1 shows that when the jump is an exact multiple of ACL the reconstruction is of worse quality. This is confirmed by PSNR values in Table 2, belonging to matrix values corresponding to

$$\frac{ACL}{gcd ( ACL , Jump )} = 1$$

in Table 1, and by comparing the reconstruction results of FIG. 5B (ACL=13, jump=13) with, e.g., reconstruction results of FIG. 5E (ACL=10, jump=7). Too small jumps are not optimal as well, as can be seen in Tables 1 and 2. Thus, reconstruction examples in FIGS. 5B to 5F demonstrate that increasing the jump does not necessarily lead to a better recovery, and in some cases, it even decreases the quality of recovery (compare, for example FIG. 5E (ACL=10, jump=7) and FIG. 5F (ACL=20, jump=27)).

In some embodiments, the optimization processes, such as the process for optimizing the mask structure, are done using ML tools, such as support vector-machine and deep neural networks.

As described above, the present invention utilizes designing of the mask pattern. In some examples, the mask pattern is designed with both sufficient randomness and spatial thickness variance. The optimal mask pattern design is selected, which provides the optimal results, i.e. best image quality with lowest required dose. As also described above, the sparsity nature of the objects (regions of interest) is considered, for example, for various organs in the human body, to determine the reduction of the dose and the number of realizations needed to enable high-quality image reconstruction. The latter may utilize the GI-based simulations combined with compressed sensing (e.g., including pseudo-inverse and sparsity constraint approaches) and/or ML tools. The structure of the mask pattern may be further optimized by obtaining the cost function, which is the number of required realizations for obtaining the object's image with the best SNR. In order to obtain the cost function, ML tools, such as Support Vector Machine and deep learning using Artificial Neural Networks, may be used.

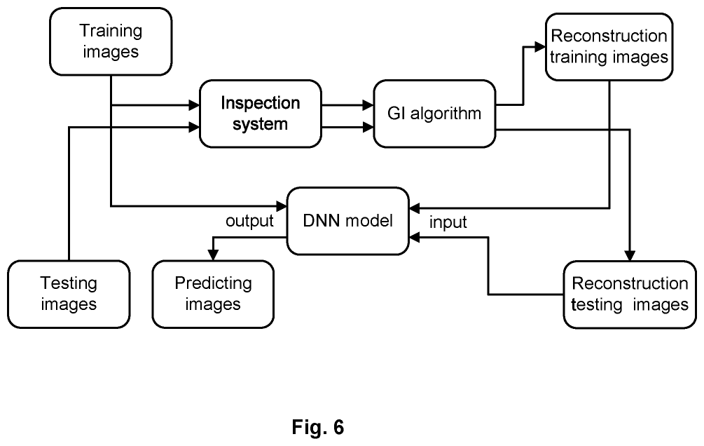

Reference is made to FIG. 6A schematically illustrating, by way of a block diagram, the use of ML tools in designing the optimal mask structure (mask pattern parameters including those relating to the thickness variation of the mask structure). A set of training images is used to design a set of training mask patterns that serve as input for a GI-based simulation which includes simulation of the inspection (i.e., imaging) system and the subsequent GI-based algorithm of image reconstruction (i.e. compressed sensing correlation-based image processing). The so-reconstructed (by simulation) set of training images, together with the original training images serve as an input to build a deep neural network (DNN) model.

As noted above, the DNN model, optimizing the mask pattern, is obtained using a cost function which is the number of needed realizations for obtaining the object's image with the best SNR. The training stage produces a set of parameters, i.e., the weights matrix of the network (representing the optimal randomness and spatial thickness variance), which minimizes a loss function over the training data (i.e. providing minimum number of realizations with the best SNR). Once the weights matrix is produced, the network can be used to either predict class labels for a new set of observations, or to perform multi-variable regression. Once the DNN model is built, a group of testing images is again subjected to simulation of the inspection (i.e., imaging) system and the subsequent GI-based algorithm of image analysis, using the optimal set of mask pattern parameters obtained during the training stage. The reconstructed testing images are input into the DNN model and the obtained predicted images are compared to the reconstructed ones via fitting (iterative procedure) to optimize the weights.

In the following, figures the results of simulations for resolution improvement are presented.

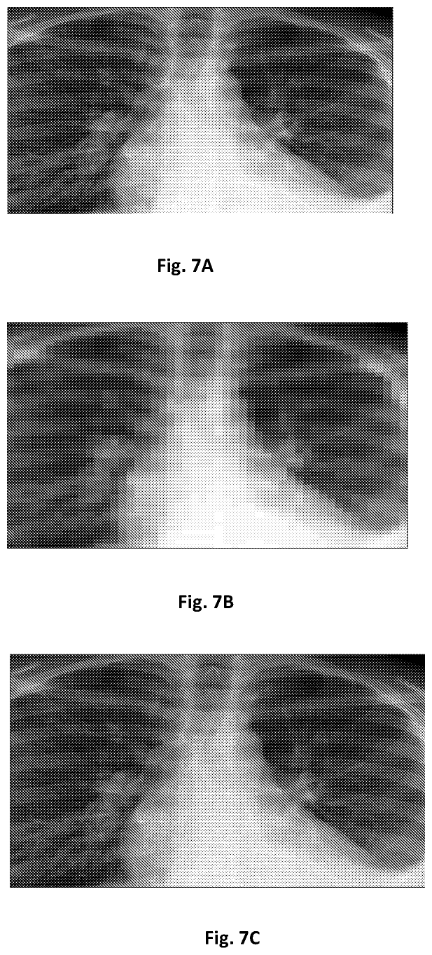

FIG. 7A shows a high-resolution chest X-ray image taken from images' bank to serve as an input for the simulation. In FIG. 7B the inventors downgraded the image of FIG. 7A to simulate lower resolution in existing imaging embodiments. Compared to the downgraded image (FIG. 7B), the techniques of GI according to the present invention, i.e., using each pixel as bucket detector to which GI is applied, provide better restoration of the image and achieve advantageous resolution (FIG. 7C).

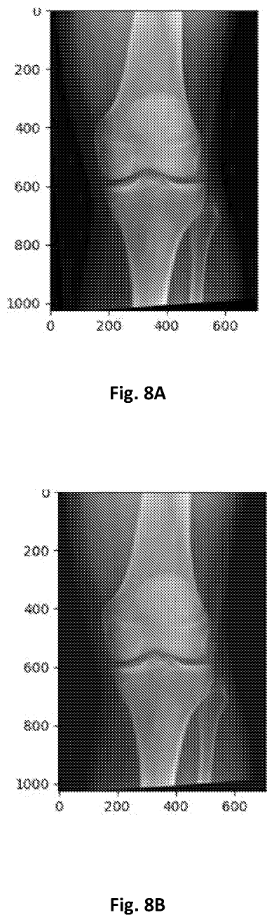

Another example, demonstrating a simulation of compression for medical imaging is shown in FIGS. 8A and 8B. FIG. 8A shows an original X-ray image taken from images' bank, taken as a reference. The inventors performed a simulation of the above-described imaging technique, similar to the one described in relation to FIGS. 7A to 7C, however, this time using a compression ratio of 30 and utilizing a DCT (Discrete Cosine Transform) basis as described in detail above. The result of the simulation is shown in FIG. 8B. It can be appreciated that the high compression factor used did not compromise the image resolution and proves that compressed sensing correlation-based imaging and DCT basis according to the techniques of the present invention may provide high-resolution images while reducing the dose of X-ray radiation due to the mask design.

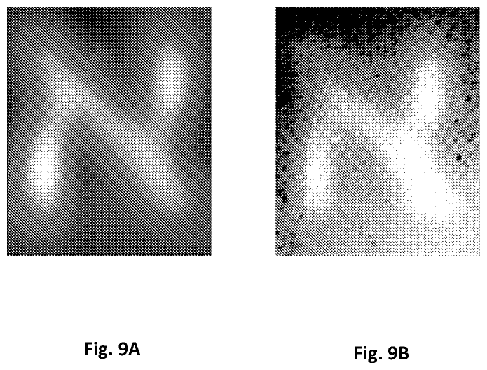

Reference is made to FIGS. 9A and 9B which illustrate the imaging of the Hebrew letter ‘Aleph’ (ϰ) using an X-ray tube source. FIG. 9A shows the image obtained by direct imaging (with no spatial intensity coding), whereas FIG. 9B was obtained by the technique of the invention utilizing the above-described mask design. Tube X-ray sources are currently used for medical imaging where radiation dose is critically controlled, as opposed to high-brightness synchrotron sources used in research applications. The image of FIG. 9B contains 5,000 pixels, while the inventors used only 500 realizations for reconstruction (the measurement time of one realization was about 1 sec). The object was the Hebrew letter Alef, which was made of a 30 nm gold on a Quartz membrane. The transmission of the gold is 98.8% at 8 keV (the image was obtained by using a lab (tube X-ray) source, which is a copper-based anode that radiates at 8.04 keV).

1. A masking assembly for use in inspecting a region of interest by creating structured high photon energy radiation to interact with the region of interest, said masking assembly comprising: at least one mask structure, the mask structure having a predetermined effective thickness t, and comprising a mask pattern formed by an arrangement of spaced-apart features of absorption properties with respect to said high photon energy radiation different from spaces between said features, said arrangement of said features along a lateral dimension of the pattern defining a predetermined effective pattern size P, wherein the features have a characteristic lateral size l providing a substantially high aspect ratio t/l.

2. The mask assembly according to claim 1, wherein the mask structure is characterized by at least one of the following:
the mask structure has a spatial modulation of thickness along the pattern within said effective thickness t;
the mask structure is a disk, said spaced-apart features of the mask pattern being arranged along a substantially circular path;
the mask pattern has one of the following configurations: the mask patter is formed by relatively highly absorbing features arranged in the spaced-apart relationship in a relatively low-absorbing substrate with respect to said high photon energy radiation; or the mask pattern is formed by relatively low-absorbing features arranged in the spaced-apart relationship in a relatively highly absorbing substrate with respect to said high photon energy radiation;
the substantially high aspect ratio t/l is at least 10;
an arrangement of the spaced-apart features of the mask pattern has one of the following configurations: a random arrangement; and an arrangement providing desired sparsity in a predetermined base selected in accordance with base of sparsity in the region of interest being inspected; and an arrangement selected in accordance with a function corresponding to discrete cosine transform.

3. (canceled)

4. The mask assembly according to claim 1, comprising a drive mechanism configured and controllably operable to implement successive lateral movements of said mask pattern with a predetermined lateral jump j to thereby enable successive inspection realizations through different segments of the mask pattern, respectively, wherein a value of said lateral jump j≠N×P, N being an integer.

5. The mask assembly according to claim 4, wherein said predetermined lateral jump j is smaller than P.

6. (canceled)

7. The mask assembly according to claim 1, wherein the mask pattern has one of the following configurations: the mask pattern is formed by relatively highly absorbing features arranged in the spaced-apart relationship in a relatively low-absorbing substrate with respect to said high photon energy radiation; or the mask pattern is formed by relatively low-absorbing features arranged in the spaced-apart relationship in a relatively highly absorbing substrate with respect to said high photon energy radiation.

8. (canceled)

9. The mask assembly according to claim 7, wherein relatively high absorbing material of the mask pattern comprises metal material composition.

10. The mask assembly according to claim 9, wherein said metal material composition comprises at least one of the following: tungsten, platinum, silver, gold and tantalum.

11. The mask assembly according to claim 1, wherein the mask pattern is formed by relatively highly absorbing features arranged in the spaced-apart relationship in a relatively low-absorbing substrate with respect to said high photon energy radiation, said mask pattern being produced by metal printing of said spaced-apart features, followed by laser milling.

12. The mask assembly according to claim 11, wherein metal material being printed comprises at least one of the following: tungsten, platinum, silver, gold and tantalum.

13. The mask assembly according to claim 8, wherein the mask pattern is formed by relatively low-absorbing features arranged in the spaced-apart relationship in a relatively highly absorbing substrate with respect to said high photon energy radiation, said mask pattern being in the form of distribution of pores in the relatively highly absorbing substrate.

14. The mask assembly according to claim 13, wherein said substrate is made from one of the following: Limestone, Mortar, AlCu, MgZn.

15. (canceled)

16. (canceled)

17. (canceled)

18. An inspection system for inspecting a region of interest, the inspection system comprising: a measurement device configured and operable to perform a plurality of successive measurements of the region of interest by implementing a corresponding plurality of interactions of the region of interest with high photon energy radiation beams having predetermined different spatially encoded intensity profiles, and a detection system comprising at least one radiation detector device successively collecting radiation responses of the region of interest to said interactions and generating measured data indicative thereof, wherein said high photon energy radiation beams having predetermined different spatially encoded intensity profiles are sequentially produced by passing an initial high photon energy radiation beam through the masking assembly of claim 1.

19. The inspection system according to claim 18, characterized by at least one of the following:
said at least one radiation detector device is configured and operable to collect said response of the region of interest comprising Compton scattering of the region of interest; and
the inspection system comprises a control system configured and operable to process said measured data, thereby enabling reconstruction of image of the region of interest from said measured data by applying thereto at least one of compressed sensing based correlation processing and/or machine learning based processing, and generate data indicative of reconstructed image of the region of interest.

20. The inspection system according to claim 18, wherein said at least one radiation detector device is configured and operable to collect said response of the region of interest comprising Compton scattering of the region of interest, said at least one radiation detector device being accommodated for collecting at least one of back propagating and forward propagating radiation of said Compton scattering of the region of interest.

21. (canceled)

22. An inspection system for inspecting a region of interest, the inspection system comprising:
a measurement device configured and operable to perform a plurality of successive measurements of the region of interest by implementing a corresponding plurality of interactions of the region of interest with high photon energy radiation beams of X- or gamma-radiation having predetermined different spatially encoded intensity profiles, and a detection system comprising at least one radiation detector device configured and operable to successively collect radiation responses of the region of interest to said interactions and generate measured data indicative thereof, wherein said at least one radiation detector device is configured and operable to collect said response of the region of interest comprising Compton scattering of the region of interest including at least one of back propagating and forward propagating radiation of said Compton scattering of the region of interest, thereby enabling reconstruction of image of the region of interest from said measured data by applying thereto at least one of compressed sensing based correlation processing and machine learning based processing.

23. The inspection system according to claim 22, characterized by one of the following: comprising a communication utility for communicating said measured data to a control system configured to perform said at least one of the compressed sensing based correlation processing and the machine learning based processing of the measure data and generate the reconstructed image of the region of interest; or comprising a control system configured to perform said at least one of the compressed sensing based correlation processing and the machine learning based processing.

24. (canceled)

25. The inspection system according to claim 22, wherein the measurement device comprises:
a) a source of the X-ray or gamma-ray radiation configured to provide an input beam of said radiation;
b) a mask assembly accommodated in a general propagation path of said input beam, the mask assembly comprising at least one mask structure being configured and controllably operated to sequentially induce different intensity fluctuations on the input beam, thereby sequentially producing beams of the X- or gamma-radiation having predetermined different spatially encoded intensity profiles, the mask structure having a predetermined effective thickness t, and comprising a mask pattern formed by an arrangement of spaced-apart features of absorption properties with respect to said high photon energy radiation different from spaces between them, said arrangement of said features along a lateral dimension of the mask pattern defining a predetermined effective pattern size P, wherein the features have a characteristic lateral size l providing a substantially high aspect ratio t/l.

26. The inspection system according to claim 25, characterized by at least one of the following:
said mask structure has a spatial modulation of thickness along the mask pattern within said effective thickness t;
said mask structure is a disk, said spaced-apart features of the mask pattern being arranged along a substantially circular path;
the system comprises a drive mechanism configured and controllably operable to implement successive lateral displacements between the general propagation path of the input beam and said mask pattern with a predetermined lateral jump j to thereby enable successive inspection realizations through different segments of the mask pattern, respectively, wherein a value of said lateral jump j≠N×P, N being an integer;
said aspect ratio t/l is at least 10;
the mask pattern has one of the following configurations: the mask pattern is formed by relatively highly absorbing features arranged in the spaced-apart relationship in a relatively low-absorbing substrate with respect to said high photon energy radiation; or the mask pattern is formed by relatively low-absorbing features arranged in the spaced-apart relationship in a relatively highly absorbing substrate with respect to said high photon energy radiation;
the mask comprises at least one of 3D printed structure, and porous materials; and
an arrangement of the spaced-apart features of the mask pattern has one of the following configurations: a random arrangement; and an arrangement providing desired sparsity in a predetermined base selected in accordance with base of sparsity in the region of interest being inspected; and an arrangement selected in accordance with a function corresponding to discrete cosine transform.

27. (canceled)

28. The inspection system according to claim 25, comprising a drive mechanism configured and controllably operable to implement successive lateral displacements between the general propagation path of the input beam and said mask pattern with a predetermined lateral jump j to thereby enable successive inspection realizations through different segments of the mask pattern, respectively, wherein a value of said lateral jump j≠N×P, N being an integer, said predetermined lateral jump j being smaller than P.

29. (canceled)

30. (canceled)

31. The inspection system according to claim 25, wherein
the mask pattern has one of the following configurations: the mask pattern is formed by relatively highly absorbing features arranged in the spaced-apart relationship in a relatively low-absorbing substrate with respect to said high photon energy radiation; or the mask pattern is formed by relatively low-absorbing features arranged in the spaced-apart relationship in a relatively highly absorbing substrate with respect to said high photon energy radiation;
the relatively highly absorbing material of the mask pattern comprises metal material composition;
the mask comprises at least one of 3D printed structure, and porous materials.

32. (canceled)

33. (canceled)

34. The inspection system according to claim 31, wherein said metal material composition comprises at least one of the following: Tungsten, Platinum, Silver, Gold and Tantalum.

35. (canceled)

36. The inspection system according to claim 25, wherein the mask pattern is formed by relatively highly absorbing features arranged in the spaced-apart relationship in a relatively low-absorbing substrate with respect to said high photon energy radiation, said mask pattern being produced by metal printing of said spaced-apart features, followed by laser milling.

37. The inspection system according to claim 36, wherein metal material being printed comprises at least one of the following: tungsten, platinum, silver, gold and tantalum.

38. The inspection system according to claim 25, wherein the mask pattern is formed by relatively low-absorbing features arranged in the spaced-apart relationship in a relatively highly absorbing substrate with respect to said high photon energy radiation, said mask pattern is-being in the form of distribution of pores in the relatively highly absorbing substrate.

39. The inspection system according to claim 38, wherein said substrate is made from one of the following: Limestone, Mortar, AlCu, MgZn.

40. (canceled)

41. (canceled)

42. (canceled)

43. A method for inspecting a region of interest by X-rays or gamma rays radiation comprising:
i) producing an input beam of X-ray or gamma-ray radiation directed along a general propagation path towards the region of interest;
ii) scanning, by the input beam on its way towards the region of inters, a mask pattern, thereby sequentially inducing different intensity fluctuations in the input beam and producing a sequence of beams of the X- or gamma-radiation having predetermined different spatially encoded intensity profiles propagating towards the region of interest to successively interact with the region of interest and induce radiation a corresponding sequence of radiation responses of the region of interest comprising Compton scattering of the region of interest, said scanning being implemented by successive lateral displacements between the general propagation path of the input beam and said mask pattern with a predetermined lateral jump j of a value satisfying a condition j≠N×P, where N is an integer and P is a predetermined effective pattern size P of the mask pattern;
iii) detecting said sequence of the radiation responses comprising at least one of back propagating and forward propagating radiation of said Compton scattering of the region of interest and generating measured data indicative thereof, thereby enabling reconstruction of image of the region of interest from said measured data by applying thereto at least one of the compressed sensing based correlation processing and the machine learning based processing.

44. The method according to claim 43, characterized by at least one of the following:
said mask pattern is made in a mask structure having a predetermined effective thickness t, the mask pattern being formed by an arrangement of spaced-apart features of different absorption properties with respect to said X-ray or gamma-ray radiation as compared with spaces between them, said arrangement of said features along a lateral dimension of the pattern defining said predetermined effective pattern size P, wherein the features have a characteristic lateral size/providing a substantially high aspect ratio t/l;
said mask pattern is made in a mask structure having a spatial modulation of thickness along the pattern within said effective thickness t;
the mask pattern comprises an arrangement of features of one of the following configurations:
arrangement providing desired sparsity in a predetermined base selected in accordance with base of sparsity in the region of interest being inspected; random arrangement of spaced-apart features;
arrangement configured in accordance with function corresponding to discrete cosine transform.

45. (canceled)

46. (canceled)

47. (canceled)

48. (canceled)
